(19) United States
(12) Patent Application Publication
(10) Pub. No.: US 2009/0287193 A1
(43) Pub. Date: Nov. 19, 2009

(54) SYSTEMS AND METHODS FOR STONE REMOVAL

(75) Inventors: Rupesh Desai, San Jose, CA (US); Steven Yee, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneous Systems, Inc., Palo Alto, CA (US)

(21) Appl. No.: 12/430,710

(22) Filed: Apr. 27, 2009

Related U.S. Application Data
(60) Provisional application No. 61/118,802, filed on Dec. 1, 2008.
(60) Provisional application No. 61/053,742, filed on May 16, 2008.

(51) Int. Cl. A61B 17/22

(52) U.S. Cl. 606/2.5; 606/127

(57) ABSTRACT

A stone removal system comprises a sweeping structure and a dilating structure. The sweeping structure is deployed on a kidney side of a urinary stone in the ureter. Should the sweeping structure be unable to remove the kidney stone, the dilating structure is placed on the bladder side of the kidney stone and used to dilate the lumen of the ureter. The dilated lumen allows the sweeping structure to more easily remove the kidney stone. Should the kidney stone still resist removal, a lithotripsy device may be introduced through the ureter and energy directed through openings in the dilation structure to fragment the stone.

### CROSS-REFERENCES TO RELATED APPLICATIONS

The present application claims the benefit of U.S. provisional patent application No. 61/053,742 (Attorney Docket No. 021807-004100US) filed on May 16, 2008, and the benefit of U.S. Provisional Patent Application No. 61/118,802 (Attorney Docket No. 021807-004200US), filed on Dec. 1, 2008, the full disclosures of which are incorporated herein by reference. The present application is related to, but does not claim the benefit of, the following commonly-owned U.S. patent application Ser. No. 12/041,241 (Attorney Docket No. 021807-004010US) filed on Mar. 3, 2008; Ser. No. 11/777,522 (Attorney Docket No. 02807-004000US), filed on Jul. 13, 2007; Ser. No. 10/886,886 (Attorney Docket No. 021807-000800US), filed on Jul. 7, 2004; and Ser. No. 11/777,515 (Attorney Docket No. 021807-000830US), filed on Jul. 13, 2007, the full disclosures of which are incorporated herein by reference.

### BACKGROUND OF THE INVENTION

1. Field of the Invention.

The present invention relates generally to medical apparatus. More particularly, the present invention relates to apparatus for treating ureters and other body lumens.

It is common for kidney stones to pass from the kidney through the ureter to the urinary bladder. While muscular peristalsis of the ureter will often pass the stones into the bladder without complication, in some instances large and/or irregularly shaped stones may become lodged within the ureter causing discomfort and potential damage to the ureter and upper collective system.

A number of ways have been proposed for dislodging such kidney stones. For example, extracorporeal shock wave lithotripsy (ESWL) can be used to break up the kidney stones but is often ineffective when the stones are present in the ureter. Moreover, ESWL can produce irregularly-shaped fragments which, while smaller than the original stone, may have sharp edges that will prevent spontaneous passage of the particles through the ureter. As an alternative to ESWL in the case of a stone or fragment impacted in the ureter, it is common practice to attempt capture, using a wire stone basket. The basket is introduced through a ureteroscope which itself is typically introduced retrograde through the urinary tract. In many cases, further lithotripsy through the scope is performed (ISWL).

It is often difficult to advance such stone baskets past the obstructing material. Attempts to pass wire baskets or other grasping apparatus past a stone lodged in the ureter also presents risk of damage to the ureter. Abrasion, stretching, or perforation of the ureter at the impaction site can cause local urine leakage or edema even if the stone or resulting debris is successfully captured; and removal of the basket with the stone may be quite difficult. In some instances, baskets containing captured stones or fragments cannot themselves be removed, and it is difficult if not impossible to release the captured stone material back into the lumen of the ureter. In those cases, the basket must often be retrieved surgically or fragmented using an endoscopic laser with the fragments removed separately. Finally, if and/or when ISWL is performed, it would be useful to have some means of stabilizing stone fragments at the treatment site, rather than letting them escape up the ureter in a retrograde direction.

As an improvement over lithotripsy and the use of baskets for collecting kidney stones and debris, it has recently been proposed to use a compacted length of material, such as a polymeric film, to form a compacted occluding structure within a ureter. The compacted length of material can be used to directly draw and remove the kidney stone from the ureter into the bladder. Alternatively, the compacted length of material can be used to contain fragments which are produced in an energy-based lithotripsy procedure. As described in prior copending applications Ser. Nos. 10/866,866; 11/777,522; and 12/041,241, the length of material can be an everting tubular member, a flat membrane which folds as an accordion structure, can or take a variety of other configurations.

While each of these approaches can be effective, if the simpler approaches such as sweeping with a compacted structure fail, then it is often necessary to start over with a more invasive or traumatic procedure, such as conventional lithotripsy. The need to successively deploy different and often single-use devices is both costly and potentially damaging to the patient. For these reasons it would be desirable to provide apparatus, methods, and systems which allow for increasing level of intervention depending on the difficulty of stone removal. In particular, it would be desirable if the systems could first utilize a simple sweeping device for drawing a stone from the ureter into the bladder. If the simple sweeping device proves ineffective, the systems should allow for removal enhancements without the need to withdraw or exchange the simple sweeping device. Such stone removal systems and protocols should allow use of the minimum level of intervention necessary for stone removal in an individual patient. Moreover, the systems and protocols should be efficient and economic as the more costly interventions, such as lithotripsy, are reserved for those patients requiring them. At least some of these objectives will be met by the inventions described below.

2. Description of the Background Art.

U.S. Pat. No. 4,295,464 describes a pair of axially spaced-apart balloons that can be used to dislodge stones from a ureter. The use of an everting sleeve composed of thin, tensilized polytetrafluoroethylene for introducing catheters to body lumens is described in U.S. Pat. Nos. 5,531,717; 5,676,688; 5,711,841; 5,897,535; 6,007,488; 6,240,968; and EP605427B1. A wire coil for preventing stone fragment movement through a body lumen during lithotripsy procedure is available under the Stone Cone tradename from Boston Scientific Corporation. See Published U.S. Application No. 2003/0120281. Copending application Ser. No. 10/794,337, filed on Mar. 5, 2004, the full disclosure of which is incorporated herein by reference, describes a sheet delivery system that could be used in performing some of the methods described herein. Various stone removal baskets and capture devices are described in U.S. Pat. Nos. 4,046,149; 4,706,671; 5,192,286; and 5,989,264. Laser lithotripsy is described in U.S. Pat. No. 5,135,534.

### BRIEF SUMMARY OF THE INVENTION

The present invention provides apparatus, systems, and methods for removing urinary or kidney stones from a ureter. The invention allows a progressive or sequenced treatment protocol where an initial attempt is made to remove the stone using a sweeping structure deployed on a kidney side of the stone. The sweeping structure will typically be a compacted, conformable length of material, as described in more detail below, but could also be other mechanically expandable structures. Should removal of the stone using the sweeping structure prove ineffective, the present invention provides for enhanced stone removal using a dilating structure which is introduced from a bladder side of the stone, typically being introduced over a shaft of the sweeping structure. The dilating structure is deployed to dilate the tract immediately below the stone and dislodge the stone, where the dislodged stone may then be drawn down through the ureter using the previously deployed sweeping structure. Should sweeping of the dislodged stone prove ineffective, protocols of the present invention allow for further intervention using a lithotripsy device introduced into the ureter while the sweeping structure remains deployed. Usually, the dilation structure will also remain deployed in order to expand the working space below the stone, but in some instances it may be desirable to remove said dilating structure prior to lithotripsy. The lithotripsy device will be used to direct energy, such as laser energy or ultrasonic energy, into the stone, typically deployed through the dilating structure which has an open scaffold or other open structure which effects the desired dilation. After the stone has been fragmented, the sweeping structure can be used to remove the stone fragments, again by drawing down the sweeping structure toward the bladder. These methods and protocols are particularly beneficial since only those patients who require the more costly and intrusive lithotripsy protocols will receive them.

In a first aspect, methods of the present invention for removing urinary stones from a ureter comprise deploying a sweeping structure on a kidney side of a urinary stone in the ureter. An open scaffold is expanded to dilate the ureter on the bladder side of the stone to dislodge or mobilize the stone. The deployed sweeping structure is then drawn to engage and sweep the stone toward the bladder. The stone may be thus withdrawn in a single sweep where both the sweeping structure and open scaffold are drawn together, typically in tandem, with the stone captured therebetween. Alternatively, the ureter may be repeatedly dilated by expanding the scaffold at different positions in the ureter as the stone is drawn sequentially toward the bladder by the sweeping structure. Typically, the expanding and drawing steps will be alternated.

While in some instances the sweeping structure may comprise balloons, cages, or other structures, it will be preferred to use a compacted length of material which provides a number of advantages. The length of material will generally be relatively flexible or soft and will be atraumatic when it is compacted within the ureter. The compacted length of material will also conform to non-circular ureter geometries as well as to the irregular shape of the kidney stone prior to disruption. Additionally, the length of material can typically be drawn to a very thin profile, thus facilitating introduction of the length of material past the kidney stone prior to compaction and enlargement. The ability to stretch and draw down the width of the material is also advantageous if it is desired to withdraw the occluding structure from the ureter and/or to release the stone or stone fragments which may have been captured in the compacted material. Such release is very difficult with a wire basket or similar structure.

Initially, the length of material will usually be positioned in the body lumen in a generally elongate or unfurled configuration. The length of material is subsequently pulled, furled, or drawn back on itself so that the material compresses or compacts into the desired occluding structure, typically forming a layered structure. The material typically comprises a polymer film, a woven fabric, a non-woven fabric, and composites and laminates thereof. Exemplary polymer materials include polytetrafluoroethylene (PTFE), polyethylene (PE), perfluoroalkoxy (PFA), polyurethane (PU), perfluoromethylvinylether (PMFA), and perfluoropropylvinylether (PPVE). Other exemplary materials include films, fabrics woven of any supple material such as nylon, polyester, silk, etc., lamination of these materials, and the like. The materials will generally be chosen so that they compress or compact into a relatively soft, non-traumatic mass of material. The compaction may be by folding, twisting, spiraling, or otherwise collapsing so that the length of the material becomes shorter and the width becomes slightly greater than the uncompacted flat film, where length is a dimension generally aligned with the axis of the body lumen and width is the dimension generally transverse to the axis when the material is in the body lumen. In the exemplary embodiments, the length of material prior to compaction is in the range from 1 cm to 10 cm, usually from 2 cm to 6 cm, and most typically from 3 cm to 5 cm. The original length will be foreshortened so that the resulting compacted mass has a width that approximates the internal diameter of the lumen in the range from 1 mm to 20 mm, usually from 2 mm to 12 mm, and preferably from 7 mm to 10 mm.

If the kidney stone resists removal using the sweeping structure alone, the open scaffold or other dilating member can then be expanded or deployed on the bladder side of the stone to dislodge or mobilize the stone. Typically, the open scaffold or other dilating structure will be positioned within a region extending 20 mm on the bladder side of the stone. The open scaffold is typically expanded by foreshortening an elongate element, such as wire, usually by mechanically drawing ends of the element together, heating the element to induce a conformational change, passing current through the element to induce a conformational change, removing a constraint from around a self-expanding structure, or the like. In the exemplary embodiments, a linear wire is foreshortened to form a radial loop, coil, cage, or the like. A radial loop which is in the form of an open tapered helix may be particularly useful for traversing the expanded dilator axially through the ureter. Alternatively, a tubular braid may be foreshortened to form an expanded braid having an open structure.

In a second aspect of the present invention, a method for urinary stone removal comprises deploying a sweeping structure on a kidney side of a urinary stone in a ureter. The sweeping structure is then drawn in an antegrade direction to pull the urinary stone from the end of the ureter and into the bladder. If the urinary stone resists being pulled by the sweeping structure, the treating physician may then introduce a dilating structure, typically coaxially over the shaft of the sweeping structure, so that it lies on the bladder side of the kidney stone. The dilating structure is then expanded to open the ureter and dislodge the stone. The sweeping structure may then be drawn to pull the dislodged urinary stone with or without the dilating structure into the bladder. Should dislodgement of the stone using the dilating structure prove insufficient, the user may then introduce a lithotripsy device into the ureter while the sweeping catheter remains in place. Energy from the lithotripsy device may then be directed into the urinary stone to fragment the stone. The sweeping structure may then be drawn to pull the fragmented stone into the bladder. Typically, the dilating structure will be left in place during use of the lithotripsy device. The open scaffold or other structure of the dilating structure allows the device to be introduced through the structure to contact the stone. Alternatively, energy from the device could be directed at the stone after removal of the open dilating structure, the prior dilation providing improved access. The use of the open dilating structure in particular, is further advantageous as it permits viewing of the stone using an endoscope or other means during removal or fragmentation.

In a third aspect of the present invention, a system for urinary stone management comprises a sweeping device including a shaft and a compactable structure at a distal end of the shaft. The system further comprises a dilation device including a body having an expandable open scaffold at a distal end of a catheter or hollow shaft. The dilation device is adapted to be advanced over the shaft of the sweeping device when the expandable structure on the sweeping device is expanded on a kidney side of a stone in the ureter. The compactable structure on the sweeping device is typically a conformal structure, but can be any of the structures described above. The scaffold structure on the dilating device may comprise any open system which permits viewing and the introduction of a catheter or other device axially therethrough. Usually, the scaffold structure will comprise a loop, coil, cage, or malecot, although in other cases it could comprise an expandable braid, typically a metal braid, and in still other cases a self-expanding cone. The open scaffold is typically expanded by foreshortening, heating, or by passing electrical current therethrough. In the exemplary embodiments, the open scaffold of the dilating device comprises a linear wire that can be foreshortened to form a loop, coil, or cage.

### BRIEF DESCRIPTION OF THE DRAWINGS

FIG. 1 illustrates a stone removal system including a sweeping structure and a dilating structure constructed in accordance with the principles of the present invention.

FIGS. 2A and 2B illustrate an exemplary sweeping structure in its pre-deployed configuration (FIG. 2A) and its deployed configuration (FIG. 2B).

FIGS. 3A and 3B illustrate an exemplary dilation structure in its pre-deployed configuration (FIG. 3A) and its deployed configuration (FIG. 3B). In particular, FIGS. 3A and 3B illustrate an elongate wire which may be foreshortened to form an open coil structure.

FIGS. 4A and 4B illustrate an alternative dilating structure in the form of a malecot in its pre-deployed configuration (FIG. 4A) and its deployed configuration (FIG. 4B).

FIGS. 5A and 5B illustrate a second alternative dilation structure comprising an expandable braid in its pre-deployed configuration (FIG. 5A) and its deployed configuration (FIG. 5B).

FIGS. 5C and 5D illustrate a third alternative dilation structure comprising a self-expanding cone held in a constraining sheath (FIG. 5C) and released from constraint to open into a deployed configuration (FIG. 5D).

FIGS. 6A-6E illustrate use of the system of FIG. 1 in dislodging a urinary stone in a ureter in accordance with the principles of the methods of the present invention.

FIG. 7 illustrates the optional deployment of a lithotripsy device for fragmenting a stone during the protocols of the present invention.

FIG. 8 illustrates deployment of the self-expanding cone dilation device for capturing a kidney stone in a further specific protocol of the present invention.

### DETAILED DESCRIPTION OF THE INVENTION

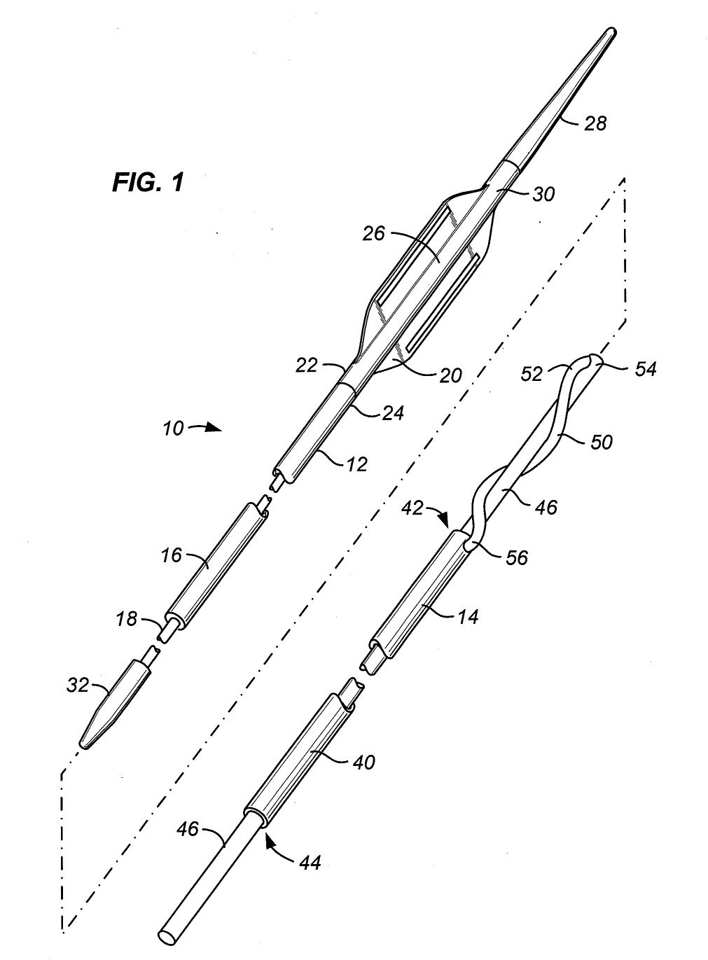

Referring now to FIG. 1, a stone removal system 10 constructed in accordance with the principles of the present invention comprises a sweeping structure 12 and a dilating structure 14. The sweeping structure 12 typically comprises a shaft 16 which slidably receives a rod 18 in a central passage thereof. A flat film 20 has a proximal end 22 attached to a distal end 24 of the shaft. The rod 18 extends through or over the length of the flat film 20, typically being positioned within a lumen 26 formed in the film. Usually, the rod 18 terminates in an atraumatic tip 28, where a distal end 30 of the film is attached to the rod just proximally of the atraumatic tip 28.

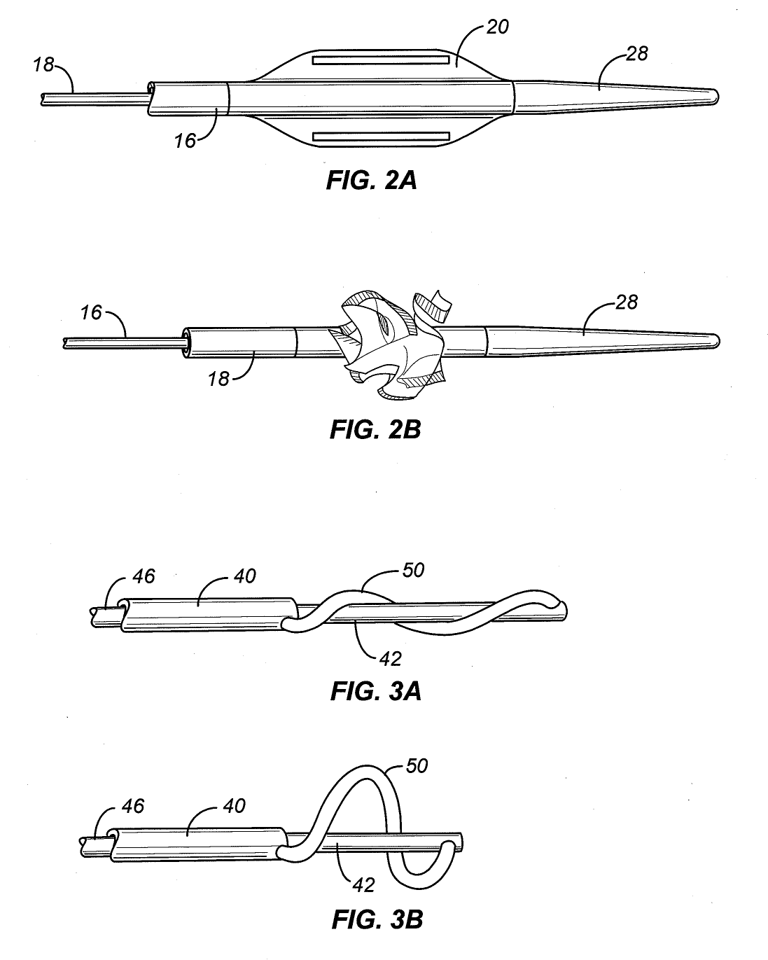

As illustrated in FIGS. 2A and 2B, the flat film 20 may be axially foreshortened and radially expanded by pulling or tensioning the rod 18 in a proximal direction relative to the shaft 16. Flat film 20 in its foreshortened, radially expanded configuration is illustrated in FIG. 2B. The film will usually have a width in the range from 7 mm to 15 mm when fully expanded.

While a useful embodiment of the sweeping structure has been illustrated in FIGS. 1, 2A, and 2B, a variety of other sweeping structures could be utilized, including in some instances balloons, expandable cages, expandable braid structures, and the like. Other specific sweeping structures which employ compacted films and other lengths of material are described in copending application Ser. Nos. 12/041,242; 11/777,522; 10/888,886; and 11/777,515, the full disclosures of which have been previously incorporated herein by reference.

The dilating structure 14 is preferably configured in FIG. 1, to be coaxially introduced over the shaft 16 of the sweeping structure 12. To facilitate such advancement, the proximal end of the sweeping structure will be configured to have a low profile, typically having a handle 32 having a diameter no greater than that of the shaft 16. Moreover, the maximum diameter of the handle and shaft will preferably be no greater than 2 mm, usually being no greater than 1 mm, requiring the dilating structure 14 to have a central lumen or passage which is only slightly greater than the outside diameter of the shaft 16 and handle 32.

The dilating structure 14 will typically comprise a hollow shaft 40 having a distal end 42 and a proximal end 44. The shaft 40 will have a central passage or lumen extending its entire length in order to slidably receive a hollow rod 46. The rod 46 extends distally of the distal end 42 and proximally of the proximal end 44 of the shaft so that a user may hold the shaft 40 and axially advance and retract the hollow rod 46 or conversely axially advance and retract the hollow shaft 40 over the hollow rod 46 using two hands. A wire element 50 is attached at a distal end 52 to a distal end 54 of the hollow rod 46 and at a proximal end 56 to the distal end 42 of the shaft 40. In this way, the hollow rod 46 may be advanced distally relative to the shaft 40 to reduce the diameter of the wire 50, as shown in FIG. 3A. By proximally drawing the rod 46 relative to the shaft 40, as shown in FIG. 3B, the wire 50 can be radially expanded into a coil or loop configuration. The coil or loop, when expanded, will typically have a diameter in the range from 10 mm to 15 mm and can act to expand a ureter or other body lumen in which it has been placed. The coiled wire 50 will expand the body lumen but leave a generally open region within the body lumen to permit access therethrough, visualization therethrough, and the like.

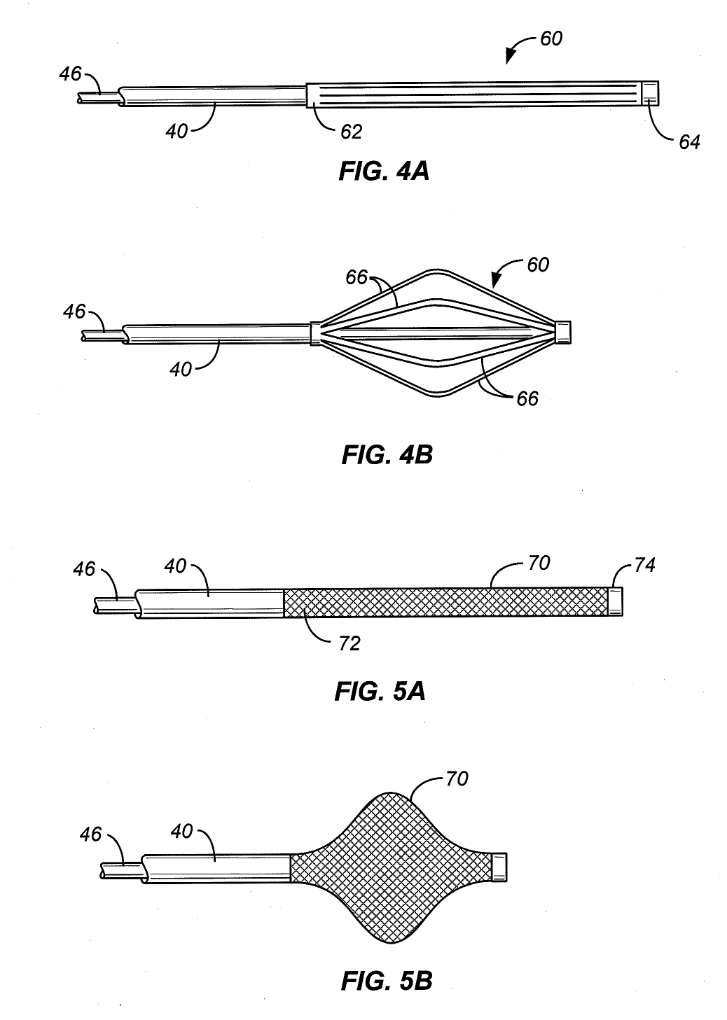

While the coiled loop embodiment of the dilating structure 14 is particularly useful, the dilating structure could comprise a variety of other expandable scaffolds or structures which can be opened or closed within the ureter or other body lumen. As shown in FIGS. 4A and 4B, a malecot structure 60 could be attached at a distal end of the shaft 40, where proximal end 62 of the malecot is attached to a distal end of the shaft and distal end 64 of the malecot is attached to the distal end of the rod 46. By axially retracting the rod 46 in a proximal direction, as shown in FIG. 4B, the malecot is expanded, forcing individual elements 66 radially outward in order to open the body lumen in which they are placed.

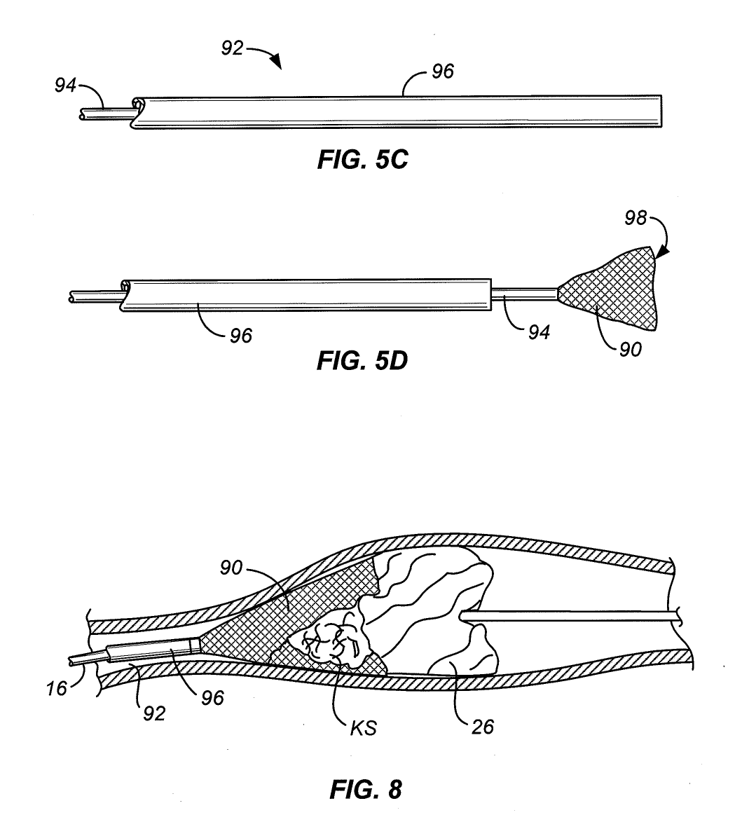

As a further alternative, the dilating structure could comprise a wire braid structure 70, as illustrated in FIGS. 5A and 5B. The wire braid structure is initially in a tubular or cylindrical configuration, as shown in FIG. 5A. The braid structure 70 is attached at its proximal end 72 to the distal end of the shaft 40, while a distal end of the rod 46 is attached to the distal end 74 of the braid. By proximally retracting the rod 46 relative to the shaft 40, as shown in FIG. 5B, the wire braid will be expanded providing the desired radial expansion about its midsection while (for relatively open braids) leaving substantial openings in an axial direction through the braid.

As shown in FIGS. 5C and 5D, a dilator structure comprising a self-expanding cone 90 can be mounted at the distal end of a dilation device 92 which further includes a hollow shaft 94 and a sheath 96. The self-expanding cone 90 is maintained in a radially constrained configuration when the sheath 96 is advanced thereover, as illustrated in FIG. 5C. By retracting the sheath 96 in a proximal direction, as shown in FIG. 5D, the cone structure 90 will self-expand so that it has an open distal end 98 for receiving and capturing a stone (or at least a leading edge of a stone) in accordance with the principles of the present invention, as described in more detail with reference to FIG. 8 below. The self-expanding cone 90 can have a variety of configurations, such as a rolled membrane, a corrugated membrane, a plurality of axial rods or elements having a mesh therebetween, or the like. Most commonly, the conical structure 90 will comprise a woven nitinol wire braid including one, two, or more layers which are attached at their proximal ends to form a proximal tip at the distal end of the shaft 94. Other conical structures which can be deployed by everting or other protocols are described in parent provisional application Ser. No. 61/118,802, the full disclosure of which is incorporated herein by reference. Typically, the shaft 94 and/or sheath 96 will be relatively stiff for introduction over the shaft of the sweeping device, but other flexible configurations could also find use.

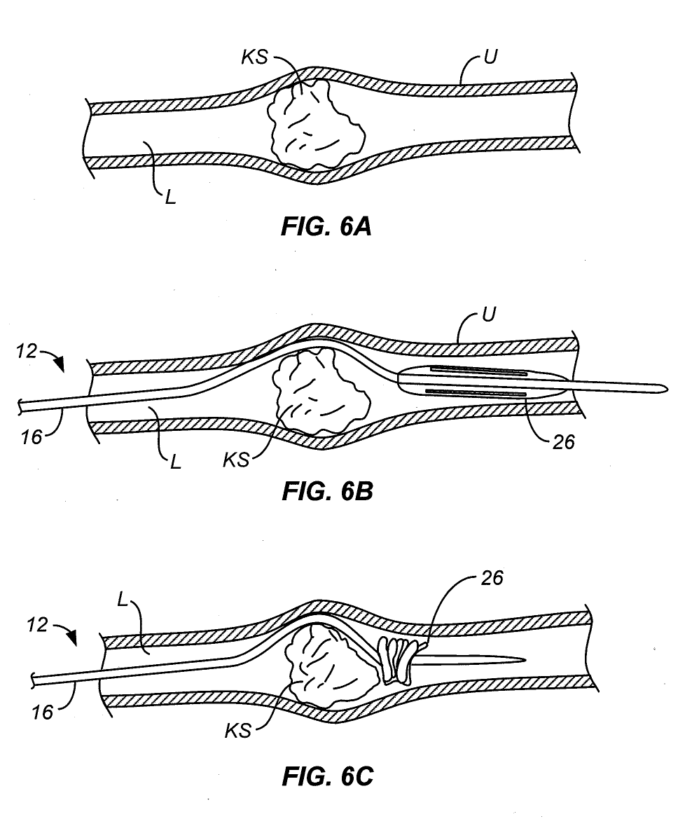

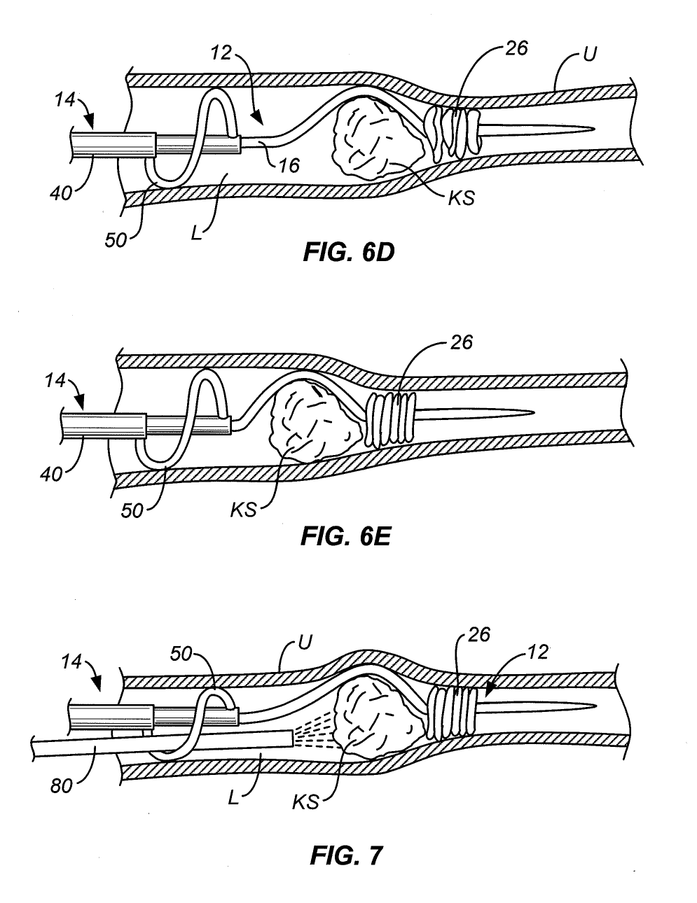

Referring now to FIGS. 6A through 6E, use of the stone removal system 10 for removing a kidney stone KS from the lumen L of a ureter U will be described. Initially, the kidney stone KS is lodged in the lumen L in such a way that it will not naturally expel into the bladder, as illustrated in FIG. 6A. Initially, the sweeping structure 12 is introduced into the ureter through cystoscopic access to the bladder in a conventional manner similar to that used for introducing guidewires, catheters, urinary stents, and the like. Flat film 26 is typically stretched or furled so that it lies very close to the shaft to facilitate advancement past the kidney stone KS, as shown in FIG. 6B. Usually, the flat film will be coated or otherwise modified in order to enhance its lubricity. Once the sweeping structure 12 has been advanced past the kidney stone KS, the rod 18 (FIG. 1) will be retracted relative to the shaft 16 so that the film is compacted into a conformable structure immediately on the bladder side of the kidney stone, as shown in FIG. 6C. Usually, the physician will then pull axially on the shaft 16 to see if the kidney stone KS can be easily dislodged and drawn into the bladder without damaging the ureter. If it can be, the procedure will be essentially complete and no further action will be required.

In the event that the kidney stone KS cannot be removed using the sweeping structure 12 alone, the methods of the present invention will further comprise dilating the lumen L of the ureter U on the bladder side of the kidney stone, as illustrated in FIG. 6D. To do so, the dilating structure 14 is coaxially advanced over the shaft 16 of the sweeping structure 12, then expanded, as shown in FIG. 6D. The expansion is caused when shaft 40 (FIG. 1) of the dilating structure is advanced relative to the rod 46 in order to radially expand the wire 50, thus opening the diameter of the lumen L of the ureter U. Usually, such a dilation will dislodge or mobilize the kidney stone KS, allowing the stone to be drawn toward the bladder using the sweeping structure, as illustrated in FIG. 6E. In some instances, a single dilation will be sufficient to allow the kidney stone to be withdrawn into the kidney, typically with the sweeping structure 12 and expanded dilating structure 14 being drawn in tandem into the bladder. Optionally, the dilating structure may be serially expanded and undeployed more than once in order to provide for sequential dilations down the ureter as the sweeping structure is used to stepwise draw the kidney stone out of the ureter.

In some instances, however, even dilation of the lumen L of the ureter U with the dilating structure 14 will not be sufficient to dislodge the stone KS. In such instances, the kidney stone KS can be further treated using a lithotripsy device 80 or other supplemental treatment, as illustrated in FIG. 7. The dilating structure may be removed to allow unfettered access to the stone, or conveniently left in place to expand the working space below the stone. Conveniently, the lithotripsy device 80 tip may be introduced through the open scaffold structure defined by the coiled wire 50. The open structure also facilitates visualization of the kidney stone. Optionally, the open scaffold structure provided by the coiled wire 50 or other dilating element allows for visualization and other access to the kidney stone which would be blocked by balloons or other dilating or treatment structures. Once the kidney stone KS has been fragmented, the sweeping structure 12 may be used to draw the fragments through the lumen L of the ureter U and into the bladder.

Referring to FIG. 8, after the flat film 26 has been expanded and pulled back against the kidney stone KS, as generally shown in FIG. 6C, dilation device 92 (FIGS. 5C and 5D) may be introduced coaxially over the shaft 16 of the sweeping structure. Once in position immediately adjacent to the bladder side of the stone, the sheath 96 will be retracted to allow the cone 90 to expand. The compacted flat film sweeping structure 26 may then be used to draw the kidney stone down into the expanded cone 90 through open end 98, as shown in FIG. 5D. As with the prior embodiments, the cone 90 and the kidney stone can be drawn distally back toward the bladder in tandem with the sweeping structure 26. Alternatively, the expanded cone 90 may be drawn distally first, followed by drawing the sweeping structure 26, and alternating such actions, in order to move the kidney stone KS into the bladder.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for urinary stone removal, said method comprising;
deploying a sweeping structure on a kidney side of a urinary stone in a ureter;
expanding an open scaffold to dilate the ureter on a bladder side of the stone to mobilize said stone; and
drawing the deployed sweeping structure to engage and sweep the stone toward the bladder.

2. A method as in claim 1, wherein the ureter is repeatedly dilated by expanding the scaffold at different positions in the ureter as the stone is drawn by the deployed sweeping structure to the bladder.

3. A method as in claim 2, wherein the expanding and drawing steps are alternated.

4. A method as in claim 1, wherein the expanding and drawing steps are performed only once with the sweeping structure, stone, and open scaffold being drawn into the bladder in tandem.

5. A method as in claim 1, wherein deploying a sweeping structure comprises compacting a length of material which when compacted conforms to the stone when the sweeping structure is drawn proximally.

6. A method as in claim 5, wherein the length of material comprises a polymeric film with a lubricious surface.

7. A method as in claim 5, wherein compacting causes the length of material to form layers on the kidney end of a shaft.

8. A method as in claim 1, wherein the open scaffold is positioned from 0 mm to 20 mm from the bladder side of the stone prior to drawing the sweeping structure forward to sweep the stone toward the bladder.

9. A method as in claim 1, wherein the open scaffold is expanded by mechanically foreshortening, heating, passing current therethrough, or by releasing a self-expanding scaffold from constraint.

10. A method as in claim 9, wherein expanding the open scaffold comprises foreshortening a linear wire to form a radial loop, coil, or cage, or foreshortening a tubular braid to form an open, expanded braid.

11. A system for urinary stone management, said system comprising:
a sweeping device including a shaft and a compactable structure at a distal end of the shaft; and
a dilation device including a body and an expandable open scaffold at a distal end of the shaft;
wherein the dilation device is adapted to be advanced over the shaft of the sweeping device when the expandable structure on the sweeping device is expanded on a kidney side of a urinary stone in a ureter.

12. A system as in claim 11, wherein the compactable structure on the sweeping device comprises a conformal structure.

13. A system as in claim 12, wherein the conformal structure comprises a length of material having a distal region attached near the distal end of the sweeping device shaft, wherein the distal end may be drawn proximally relative to the stone to compact the length of material and move the compacted material against the urinary stone.

14. A system as in claim 13, wherein the length of material comprises a strip, sleeve, ribbon, or tube.

15. A system as in claim 14, wherein the material is selected from the group consisting of polymer films, woven fabrics, non-woven fabrics, and composites and laminates thereof.

16. A system as in claim 13, wherein the shaft comprises a tension member which is positioned through a substantially continuous passage formed in, on, or through the length of material.

17. A system as in claim 16, wherein the shaft is attached at a distal end of the length of material.

18. A system as in claim 11, wherein the shaft of the sweeping device has sufficient column strength to advance the length of material up the body lumen.

19. A system as in claim 18, wherein the tension member comprises a guide wire structure.

20. A system as in claim 11, wherein the sweeping device comprises:
a tension member having a proximal end and a distal end;
an elongate shaft having a guide structure along at least a distal portion thereof for receiving the tension member and permitting the tension member to shift between a distally extended position and a proximally retracted position relative to the shaft; and
a flat film having an axial receptacle for receiving a distal portion of the tension member wherein a distal end of the film is attached to a distal location on the tension member and a proximal end of the film is attached to a distal end of the elongate shaft,
wherein proximally translating the tension member relative to the shaft compacts the flat film and distally translating the tension member relative to the shaft stretches the film.

21. A system as in claim 11, wherein the scaffold on the dilation device comprises a loop, a coil, a cage, or a malecot.

22. A system as in claim 11, wherein the open scaffold on the dilation device is expanded by foreshortening, heating, or by passing an electrical current therethrough.

23. A system as in claim 11, wherein the expandable structure on the open scaffold of the dilation device comprises a linear wire that can be foreshortened to form a radial loop, coil, or cage.

24. A system as in claim 23, wherein the coil is helical with tapered end oriented towards the bladder.

25. A system as in claim 11, wherein the expandable open scaffold comprises a tubular braid which can be foreshortened to form an open structure.

26. A system as in claim 11, wherein the scaffold on the dilation device comprises a self-expanding cone.

27. A system as in claim 26, wherein the self-expanding cone is formed from a shape memory material and has an unconstrained radially expanded configuration and a reduced width constrained configuration.

28. A system as in claim 27, wherein the dilation device further comprises a sheath which can be advanced over the cone to constrain the cone in its reduced width configuration and which can be retracted to release the cone to its radially expanded configuration.

29. A method for urinary stone removal, said method comprising:
deploying a sweeping structure on a kidney side of a urinary stone in a ureter;
drawing on the sweeping structure to pull the urinary stone into a bladder at an end of the ureter;
if the urinary stone being pulled by the sweeping structure resists being pulled into the bladder,
introduce a dilating structure over the sweeping structure into the ureter;
expand a dilating structure on a bladder side of the stone to dislodge the stone; and
draw on the sweeping structure to pull the dislodged urinary stone into the bladder.

30. A method as in claim 29, further comprising:
if the dislodged urinary stone resists being pulled into the bladder, then perform the following;
introduce a lithotripsy device into the ureter while the sweeping catheter remains in place; and
direct energy from the lithotripsy device into the urinary stone to fracture said stone; and
draw the sweeping structure to pull the fragmented stone into the bladder.

31. A method as in claim 27, wherein the dilating structure is open and the lithotripsy device tip is introduced through the open dilating structure.
